(19) United States
(12) Patent Application Publication
(10) Pub. No.: US 2012/0035727 A1
(43) Pub. Date: Feb. 9, 2012

(54) SURGICAL IMPLANT DEVICE FOR THE TRANSLATION AND FUSION OF A FACET JOINT OF THE SPINE

(76) Inventor: Robert E. LINS, Boca Raton, FL (US)

(21) Appl. No.: 13/276,058

(22) Filed: Oct. 18, 2011

Related U.S. Application Data
(63) Continuation-in-part of application No. 12/875,374, filed on Sep. 3, 2010.
(60) Provisional application No. 61/239,594, filed on Sep. 3, 2009.

(51) Int. Cl. A61F 2/44

(52) U.S. Cl. 623/17.11

(57) ABSTRACT

The present invention provides, among other things, a surgical device for the translation/distraction and subsequent stabilization/fusion of a facet joint of a spine, including: a post that is selectively disposed partially between articulating surfaces of the facet joint; and a keel structure that is selectively disposed about the post and impacted into the articulating surfaces of the facet joint, wherein, when the keel structure is selectively rotated about the post, the articulating surfaces of the facet joint are moved with respect to one another.

### CROSS-REFERENCE TO RELATED APPLICATIONS

The present patent application/patent is a continuation-in-part of U.S. patent application Ser. No. 12/875,374, filed on Sep. 3, 2010, and entitled “SURGICAL IMPLANT DEVICE FOR THE TRANSLATION AND FUSION OF A FACET JOINT OF THE SPINE,” which claims the benefit of priority of U.S. Provisional Patent Application No. 61/239,594, filed on Sep. 3, 2009, and entitled “SURGICAL IMPLANT DEVICE FOR THE TRANSLATION AND FUSION OF A FACET JOINT OF THE SPINE,” the contents of both of which are incorporated in full by reference herein.

### FIELD OF THE INVENTION

The present invention relates generally to a novel surgical implant device for treating spinal stenosis, facet arthropathy, degenerative disc disease, and the like. More specifically, the present invention relates to a novel surgical implant device for the translation/distraction and subsequent stabilization/fusion of a facet joint of the spine in the treatment of such conditions.

### BACKGROUND OF THE INVENTION

There are a variety of conventional surgical implant devices and methodologies for stabilizing/fusing a facet joint of the spine. Most of these devices and methodologies involve drilling between and across the articulating surfaces of the facet joint while un-translated/non-distracted and inserting a plug or other stabilization structure in the drilled hole(s). Some of these device and methodologies involve placing a bolt or other retention structure through (i.e. substantially perpendicularly across) or about the articulating surfaces of the facet joint while un-translated/non-distracted. These conventional surgical implant devices and methodologies, however, suffer from a number of significant shortcomings and often fail to adequately address patient symptoms.

### BRIEF SUMMARY OF THE INVENTION

It is desirable, in many applications, to translate/distract the facet joint before stabilizing/fusing it, especially in the lumbar spine. This may be accomplished, for example, by placing a surgical implant device in the facet joint and rotating it, thus displacing the articulating surfaces of the facet joint with respect to one another with a translation motion and/or a distraction motion. Such a procedure may be carried out both left and right at each level of the spine. Further, all procedures of the present invention may be performed open, through a portal tube or the like, or percutaneously.

Advantageously, such displacement increases the size of the foramen, where the nerve roots exit the central spinal canal, thus addressing foraminal stenosis, which may cause leg symptoms. Such displacement also addresses central spinal canal stenosis by unbuckling or stretching out the redundant ligamentum flavum which connect each spinal segment posteriorly. Such displacement further unloads the posterior aspect of the intervertebral disc posteriorly, and may be used to address underlying degenerative disc disease, in addition to lumbar spinal stenosis, facet arthropathy (i.e. facet arthritis), and the like.

The surgical procedures of the present invention may be performed percutaneously or through two small incisions on the back, one on each side. An elongated device with a sharp point and a plurality of concentrically-arranged friction structures, for example, is inserted into the facet joint and rotated, thus providing translation/distraction. A retention sleeve is then slid down the elongated device and into or adjacent to and engaging the facet joint to maintain the facet joint in translation/distraction while the elongated device is removed. Subsequently or alternatively, a hole is drilled between and across the articulating surfaces of the facet joint through the retention sleeve and a plug or other novel surgical implant device is tamped into the hole to maintain the facet joint in translation/distraction. This later function may be accomplished using the retention sleeve itself, in the case that it is simply a toothed retention washer or the like. Alternatively, the novel surgical implant device may be inserted into the facet joint, rotated to translate/distract the facet joint, and then remain in place itself (optionally after additional seating) to hold the facet joint in the desired configuration. This surgical implant device may be a detachable end portion of the elongated device, for example.

Alternatively, after translating/distracting, a stellate/snowflake-shaped tamp may be impacted into and across the facet joint to create an outline for a serrated surgical implant device to subsequently be impacted into this outline. This provides an interference fit and prevents unwinding of the facet joint. Various surgical implant device configurations are contemplated, illustrated, and described herein, including various friction structures and various other structures that aide in the translation/distraction of the facet joint and variously fill the “gap” therein.

The goal of the present invention is to stabilize/fuse the facet joint in a desirable configuration that alleviates a given physical ailment or condition. The various surgical implant devices of the present invention may be made of machined allograft (i.e. bony) material, a surgically-implantable polymeric material, a surgically-implantable ceramic material, a surgically-implantable metallic material, etc., and may include one or more holes or pores for the impaction of another material that promotes the fusion of the superior and inferior facets of a facet joint.

In one exemplary embodiment, the present invention provides a surgical implant device for the translation/distraction and subsequent stabilization/fusion of a facet joint of a spine, including: a body that is selectively disposed at least partially between articulating surfaces of the facet joint; and one or more protruding structures disposed about the body, wherein, when the body is selectively rotated, the one or more protruding surfaces are configured to engage the articulating surfaces of the facet joint and move them with respect to one another. Optionally, the surgical implant device also includes a joint-spanning structure coupled to the body, wherein the joint-spanning structure is configured to substantially fill a space between the articulating surfaces of the facet joint and hold it in a moved configuration.

In another exemplary embodiment, the present invention provides a surgical implant method for the translation/distraction and subsequent stabilization/fusion of a facet joint of a spine, including: selectively disposing a body at least partially between articulating surfaces of the facet joint; and selectively rotating the body such that one or more protruding structures disposed about the body engage the articulating surfaces of the facet joint and move them with respect to one another. Optionally, the method also includes providing a joint-spanning structure coupled to the body, wherein the joint-spanning structure is configured to substantially fill a space between the articulating surfaces of the facet joint and hold it in a moved configuration.

In a further exemplary embodiment, the present invention provides a surgical implant system for the translation/distraction and subsequent stabilization/fusion of a facet joint of a spine, including: a tool that is selectively disposed at least partially between articulating surfaces of the facet joint; and one or more protruding structures disposed about the tool, wherein, when the tool is selectively rotated, the one or more protruding surfaces are configured to engage the articulating surfaces of the facet joint and move them with respect to one another. The surgical implant system also includes a sheath disposed about the tool, wherein the sheath is selectively slid down the tool to engage the facet joint to maintain the facet joint in a moved configuration while the tool is removed. The surgical implant system further includes a surgical implant device that is selectively disposed at least partially between articulating surfaces of the facet joint to maintain the facet joint in the moved configuration while the sheath is removed.

In a still further exemplary embodiment, the present invention provides a surgical implant method for the translation/distraction and subsequent stabilization/fusion of a facet joint of a spine, including: moving a first articulating surface of the facet joint with respect to a second articulating surface of the facet joint; forming a cut-away portion of each of the articulating surfaces of the facet joint; and disposing a surgical implant device in the cut-away portion of each of the articulating surfaces of the facet joint to prevent unwinding thereof.

In a still further exemplary embodiment, the present invention provides a surgical device for the translation/distraction and subsequent stabilization/fusion of a facet joint of a spine, including: a post that is selectively disposed partially between articulating surfaces of the facet joint; and a keel structure that is selectively disposed about the post and impacted into the articulating surfaces of the facet joint, wherein, when the keel structure is selectively rotated about the post, the articulating surfaces of the facet joint are moved with respect to one another. Optionally, the keel structure has a substantially planar configuration. Preferably, the keel structure has a sharpened lower portion.

In a still further exemplary embodiment, the present invention provides a surgical system for the translation/distraction and subsequent stabilization/fusion of a facet joint of a spine, including: a portal tube defining a first cut-away portion, wherein, when placed, the first cut-away portion provides surgical access to adjacent spinous processes of the spine. Optionally, the portal tube further defines a second cut-away portion disposed substantially opposite the first cut-away portion, wherein, when placed, the second cut-away portion provides surgical access to an associated facet joint of the spine. The surgical system also includes a distraction device that is selectively disposed within the tube, through the first cut-away portion, and between the adjacent spinous processes. The surgical system further includes a facet joint implant that is selectively disposed within the tube, through the second cut-away portion, and within a receiving hole formed in the facet joint.

In a still further exemplary embodiment, the present invention provides a surgical method for the translation/distraction and subsequent stabilization/fusion of a facet joint of a spine, including: through a portal tube, disposing a distraction device between adjacent spinous processes of the spine and distracting the adjacent spinous processes; forming a hole across the facet joint of the spine; and inserting a facet implant device in the hole formed across the facet joint. The surgical method also includes removing the distraction device and performing a discectomy. The surgical method further includes replacing the distraction device and again distracting the adjacent spinous processes.

In a still further exemplary embodiment, the present invention provides a surgical device for the translation/distraction and subsequent stabilization/fusion of a facet joint of a spine, including: a rotation device that is selectively disposed at least partially between and engages articulating surfaces of the facet joint, wherein the rotation device is operable for rotating and translating the articulating surfaces of the facet joint; and a translation sheath disposed about the rotation device that is selectively mated with a superior facet and an inferior facet of the facet joint, wherein the translation sheath is operable for rotating and further translating the articulating surfaces of the facet joint. The translation sheath comprises a plurality of raised structures disposed about a circumference thereof. The translation sheath comprises a plurality of spike structures disposed about a circumference thereof.

In a still further exemplary embodiment, the present invention provides a surgical implant device for the stabilization/fusion of a facet joint of a spine, including: a liquid or semi-solid polymeric material that is disposed in a cut-away portion of each articulating surface of the facet joint and subsequently allowed to harden to a solid to prevent movement thereof. Optionally, the surgical implant device also includes a solid material that is disposed within or about the liquid or semi-solid polymeric material within the cut-away portion of each articulating surface of the facet joint to prevent movement thereof.

### BRIEF DESCRIPTION OF THE DRAWINGS

The present invention is illustrated and described herein with reference to the various drawings, in which like reference numbers are used to denote like device components/method steps, as appropriate, and in which:

FIG. 1 is a schematic diagram illustrating one exemplary embodiment of the facet implant device of the present invention, and one exemplary embodiment of the facet translation/fusion method of the present invention;

FIG. 2 is a schematic diagram illustrating another exemplary embodiment of the facet implant device of the present invention, and another exemplary embodiment of the facet translation/fusion method of the present invention;

FIG. 3 is a schematic diagram illustrating one exemplary embodiment of the facet implant device of the present invention;

FIG. 4 is a schematic diagram illustrating another exemplary embodiment of the facet implant device of the present invention;

FIG. 5 is a schematic diagram illustrating a further exemplary embodiment of the facet implant device of the present invention;

FIG. 6 is a schematic diagram illustrating a still further exemplary embodiment of the facet implant device of the present invention;

FIG. 7 is a schematic diagram illustrating a still further exemplary embodiment of the facet implant device of the present invention;

FIG. 8 is a schematic diagram illustrating a still further exemplary embodiment of the facet implant device of the present invention;

FIG. 9 is a schematic diagram illustrating a still further exemplary embodiment of the facet implant device of the present invention;

FIG. 10 is a perspective diagram illustrating one exemplary embodiment of the facet implant device tool assembly of the present invention;

FIG. 11 is a series of schematic diagrams illustrating another exemplary embodiment of the facet translation/fusion method of the present invention;

FIG. 12 is a series of schematic diagrams illustrating a still further exemplary embodiment of the facet implant device of the present invention;

FIG. 13 is a schematic diagram illustrating one exemplary embodiment of the translation sheath of the present invention;

FIG. 14 is a schematic diagram illustrating one exemplary embodiment of the facet portal system of the present invention;

FIG. 15 is another schematic diagram illustrating one exemplary embodiment of the facet portal system of the present invention;

FIG. 16 is a further schematic diagram illustrating one exemplary embodiment of the facet portal system of the present invention;

FIG. 17 is a schematic diagram illustrating one exemplary embodiment of the facet keel device of the present invention; and

FIG. 18 is another schematic diagram illustrating one exemplary embodiment of the facet keel device of the present invention.

### DETAILED DESCRIPTION OF THE INVENTION

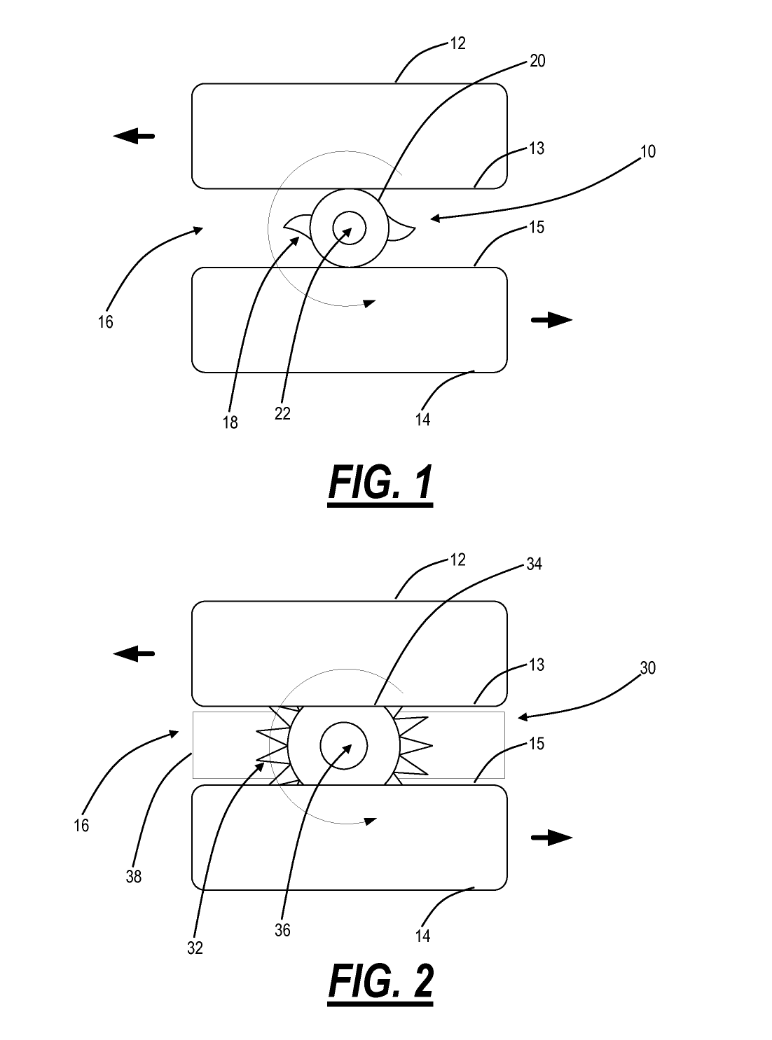

Referring to FIG. 1, in one exemplary embodiment of the present invention, the facet implant device 10 is disposed between the superior facet 12 and the inferior facet 14 of the facet joint 16 of a spine. The facet implant device 10 includes one or more protruding structures 18 or friction surfaces that engage the articulating surfaces 13, 15 of the facet joint 16 when the facet implant device 10 is rotated in the facet joint 16, the protruding structures 18 first contacting the articulating surfaces 13, 15, then grabbing them, then translating/distracting them with respect to one another, and then holding them securely in this translated/distracted configuration. In the embodiment illustrated, the facet implant device 10 includes a substantially-cylindrical body 20 and two or four substantially-triangular or fin-like protruding structures 18 that have sharp surfaces or edges for engaging the articulating surfaces 13, 15, although other suitable assemblies are contemplated herein. The facet implant device 10 has over all dimensions on the order of several millimeters, and may be made of machined allograft (i.e. bony) material, a surgically-implantable polymeric material, a surgically-implantable ceramic material, a surgically-implantable metallic material, etc. The facet implant device 10 may include one or more holes or pores 22 along its major axis and/or perpendicular to its major axis through the body 20 thereof for the impaction of another material that promotes the fusion of the superior and inferior facets 12, 14 of the facet joint 16. In addition, the body 20 of the facet implant device 10 may have a threaded portion or other attachment means for receiving one or more tools by which it is tamped into the facet joint 16 and/or rotated. The implants and devices of the present invention may be placed in or between cut-away, roughened, otherwise prepared surfaces, or otherwise unprepared surfaces of the bony structures involved in all embodiments, provided that desirable and engagement therewith is achieved. In this respect, hole, bore, cut-away portion, roughened portion, prepared portion, receiving portion, and the like are all intended to be essentially synonymous and interchangeable.

Referring to FIG. 2, in another exemplary embodiment of the present invention, the facet implant device 30 is disposed between the superior facet 12 and the inferior facet 14 of the facet joint 16 of a spine. The facet implant device 30 includes one or more protruding structures 32 or friction surfaces that engage the articulating surfaces 13, 15 of the facet joint 16 when the facet implant device 30, or a portion thereof, is rotated in the facet joint 16, the protruding structures 32 first contacting the articulating surfaces 13, 15, then grabbing them, then translating/distracting them with respect to one another, and then holding them securely in this translated/distracted configuration. In the embodiment illustrated, the facet implant device 30 includes a substantially-cylindrical body 34 and a plurality of substantially-triangular or tooth-like protruding structures 32 that have sharp surfaces or edges for engaging the articulating surfaces 13, 15, although other suitable assemblies are contemplated herein. The facet implant device 30 has over all dimensions on the order of several millimeters, and may be made of machined allograft (i.e. bony) material, a surgically-implantable polymeric material, a surgically-implantable ceramic material, a surgically-implantable metallic material, etc. The facet implant device 30 may include one or more holes or pores 36 along its major axis and/or perpendicular to its major axis through the body 34 thereof for the impaction of another material that promotes the fusion of the superior and inferior facets 12, 14 of the facet joint 16. In addition, the body 34 of the facet implant device 30 may have a threaded portion or other attachment means for receiving one or more tools by which it is tamped into the facet joint 16 and/or rotated. In the embodiment illustrated, the facet implant device 30 also includes a joint-spanning structure 38 coupled to the body 34. This joint-spanning structure 38 may or may not rotate with the body 34 when it is rotated in the facet joint 16 and, in any case, is used to substantially fill the facet joint 16, providing friction surfaces that prevent the articulating surfaces 13, 15 from sliding with respect to one another once translation/distraction has been achieved. Accordingly, the joint-spanning structure 38 may have a substantially-rectangular or other suitable shape and a thickness on the order of several millimeters.

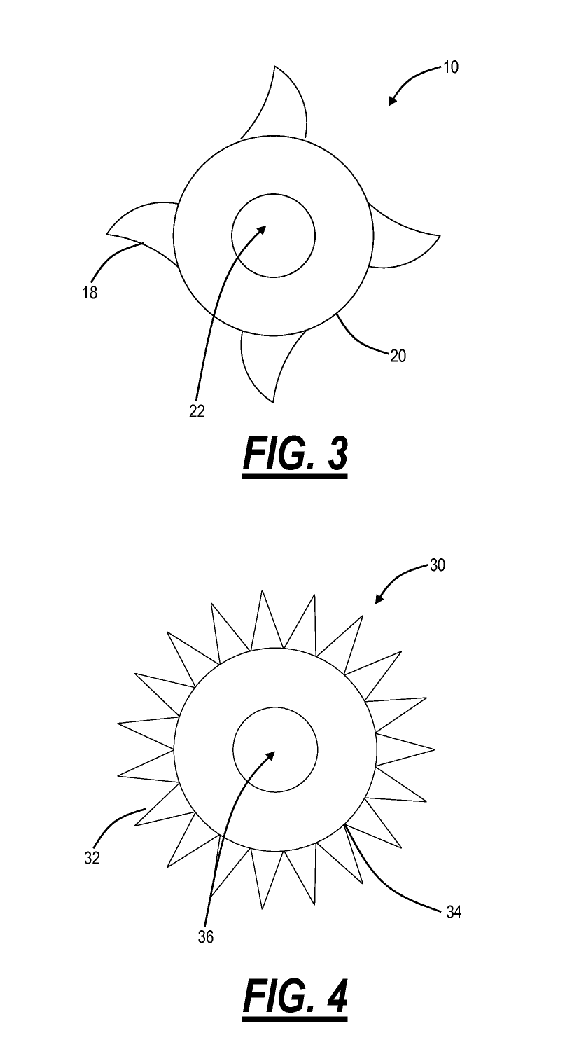

Referring to FIG. 3, in one exemplary embodiment of the present invention, the facet implant device 10 includes one or more protruding structures 18 or friction surfaces that engage the articulating surfaces 13, 15 (FIGS. 1 and 2) of the facet joint 16 (FIGS. 1 and 2) when the facet implant device 10 is rotated in the facet joint 16, the protruding structures 18 first contacting the articulating surfaces 13, 15, then grabbing them, then translating/distracting them with respect to one another, and then holding them securely in this translated/distracted configuration. In the embodiment illustrated, the facet implant device 10 includes a substantially-cylindrical body 20 and two or four substantially-triangular or fin-like protruding structures 18 that have sharp surfaces or edges for engaging the articulating surfaces 13, 15, although other suitable assemblies are contemplated herein. The facet implant device 10 has over all dimensions on the order of several millimeters, and may be made of machined allograft (i.e. bony) material, a surgically-implantable polymeric material, a surgically-implantable ceramic material, a surgically-implantable metallic material, etc. The facet implant device 10 may include one or more holes or pores 22 along its major axis and/or perpendicular to its major axis through the body 20 thereof for the impaction of another material that promotes the fusion of the superior and inferior facets 12, 14 (FIGS. 1 and 2) of the facet joint 16. In addition, the body 20 of the facet implant device 10 may have a threaded portion or other attachment means for receiving one or more tools by which it is tamped into the facet joint 16 and/or rotated.

Referring to FIG. 4, in another exemplary embodiment of the present invention, the facet implant device 30 includes one or more protruding structures 32 or friction surfaces that engage the articulating surfaces 13, 15 (FIGS. 1 and 2) of the facet joint 16 (FIGS. 1 and 2) when the facet implant device 30 is rotated in the facet joint 16, the protruding structures 32 first contacting the articulating surfaces 13, 15, then grabbing them, then translating/distracting them with respect to one another, and then holding them securely in this translated/distracted configuration. In the embodiment illustrated, the facet implant device 30 includes a substantially-cylindrical body 34 and a plurality of substantially-triangular or tooth-like protruding structures 32 that have sharp surfaces or edges for engaging the articulating surfaces 13, 15, although other suitable assemblies are contemplated herein. The facet implant device 30 has over all dimensions on the order of several millimeters, and may be made of machined allograft (i.e. bony) material, a surgically-implantable polymeric material, a surgically-implantable ceramic material, a surgically-implantable metallic material, etc. The facet implant device 30 may include one or more holes or pores 36 along its major axis and/or perpendicular to its major axis through the body 34 thereof for the impaction of another material that promotes the fusion of the superior and inferior facets 12, 14 (FIGS. 1 and 2) of the facet joint 16. In addition, the body 34 of the facet implant device 30 may have a threaded portion or other attachment means for receiving one or more tools by which it is tamped into the facet joint 16 and/or rotated.

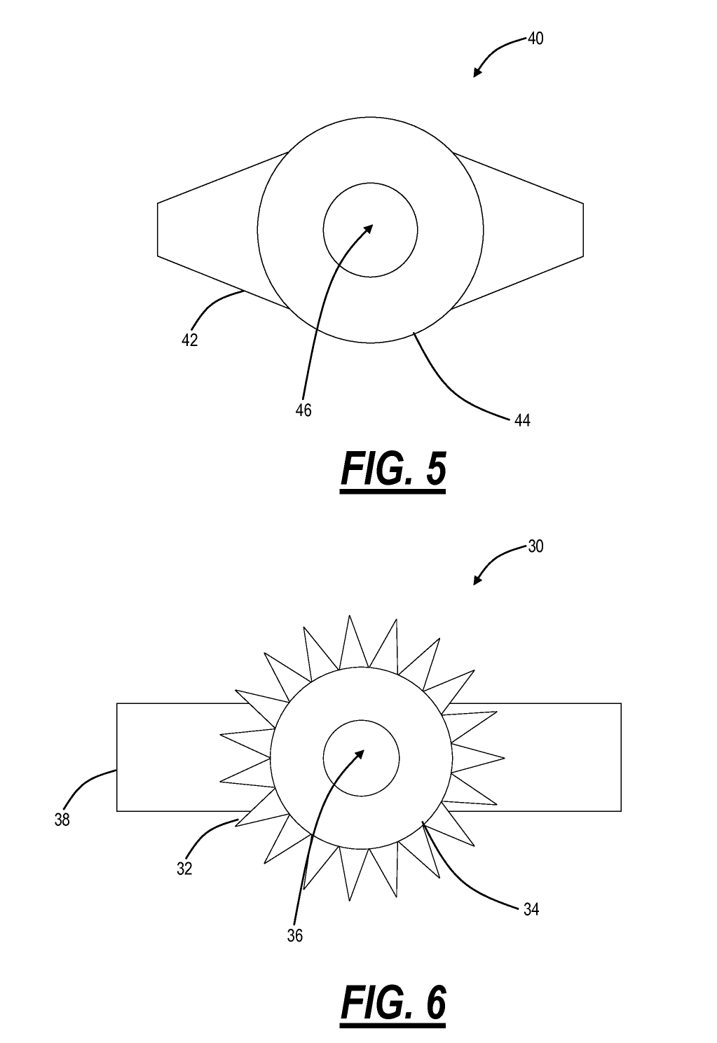

Referring to FIG. 5, in a further exemplary embodiment of the present invention, the facet implant device 40 includes one or more protruding structures 42 or friction surfaces that engage the articulating surfaces 13, 15 (FIGS. 1 and 2) of the facet joint 16 (FIGS. 1 and 2) when the facet implant device 40 is rotated in the facet joint 16, the protruding structures 42 first contacting the articulating surfaces 13, 15, then grabbing them, then translating/distracting them with respect to one another, and then holding them securely in this translated/distracted configuration. In the embodiment illustrated, the facet implant device 40 includes a substantially-cylindrical body 44 and two substantially-trapezoidal or fin-like protruding structures 42 that have sharp surfaces or edges for engaging the articulating surfaces 13, 15, although other suitable assemblies are contemplated herein. The facet implant device 40 has over all dimensions on the order of several millimeters, and may be made of machined allograft (i.e. bony) material, a surgically-implantable polymeric material, a surgically-implantable ceramic material, a surgically-implantable metallic material, etc. The facet implant device 40 may include one or more holes or pores 46 along its major axis and/or perpendicular to its major axis through the body 44 thereof for the impaction of another material that promotes the fusion of the superior and inferior facets 12, 14 (FIGS. 1 and 2) of the facet joint 16. In addition, the body 44 of the facet implant device 40 may have a threaded portion or other attachment means for receiving one or more tools by which it is tamped into the facet joint 16 and/or rotated.

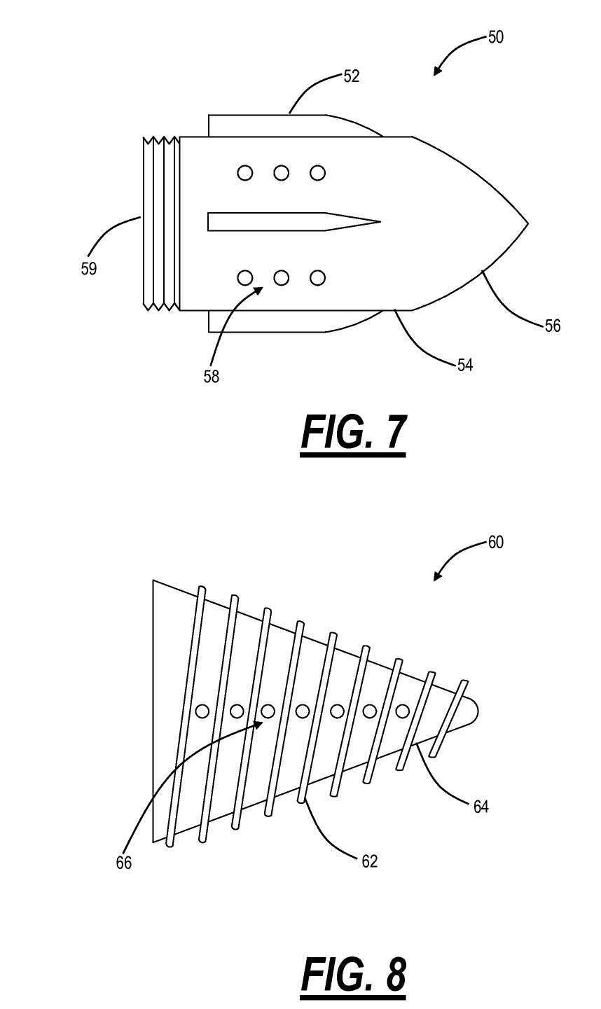

Referring to FIG. 6, in a still further exemplary embodiment of the present invention, the facet implant device 30 includes one or more protruding structures 32 or friction surfaces that engage the articulating surfaces 13, 15 (FIGS. 1 and 2) of the facet joint 16 (FIGS. 1 and 2) when the facet implant device 30, or a portion thereof, is rotated in the facet joint 16, the protruding structures 32 first contacting the articulating surfaces 13, 15, then grabbing them, then translating/distracting them with respect to one another, and then holding them securely in this translated/distracted configuration. In the embodiment illustrated, the facet implant device 30 includes a substantially-cylindrical body 34 and a plurality of substantially-triangular or tooth-like protruding structures 32 that have sharp surfaces or edges for engaging the articulating surfaces 13, 15, although other suitable assemblies are contemplated herein. The facet implant device 30 has over all dimensions on the order of several millimeters, and may be made of machined allograft (i.e. bony) material, a surgically-implantable polymeric material, a surgically-implantable ceramic material, a surgically-implantable metallic material, etc. The facet implant device 30 may include one or more holes or pores 36 along its major axis and/or perpendicular to its major axis through the body 34 thereof for the impaction of another material that promotes the fusion of the superior and inferior facets 12, 14 (FIGS. 1 and 2) of the facet joint 16. In addition, the body 34 of the facet implant device 30 may have a threaded portion or other attachment means for receiving one or more tools by which it is tamped into the facet joint 16 and/or rotated. In the embodiment illustrated, the facet implant device 30 also includes a joint-spanning structure 38 coupled to the body 34. This joint-spanning structure 38 may or may not rotate with the body 34 when it is rotated in the facet joint 16 and, in any case, is used to substantially fill the facet joint 16, providing friction surfaces that prevent the articulating surfaces 13, 15 from sliding with respect to one another once translation/distraction has been achieved. Accordingly, the joint-spanning structure 38 may have a substantially-rectangular or other suitable shape and a thickness on the order of several millimeters. In an alternative embodiment, neither the body 34 or the joint-spanning structure 38 may be rotated, but may simply be used to fill the facet joint 16 and any manufactured recesses and keep the facet joint 16 from unwinding. In this embodiment, only the upper and lower portions of the body 34 need have teeth or fins 32, for example.

Referring to FIG. 7, in a still further exemplary embodiment of the present invention, the facet implant device 50 includes one or more protruding structures 52 or friction surfaces that engage the articulating surfaces 13, 15 (FIGS. 1 and 2) of the facet joint 16 (FIGS. 1 and 2) when the facet implant device 50 is rotated in the facet joint 16, the protruding structures 52 first contacting the articulating surfaces 13, 15, then grabbing them, then translating/distracting them with respect to one another, and then holding them securely in this translated/distracted configuration. In the embodiment illustrated, the facet implant device 50 includes a substantially-cylindrical body 54, a substantially-conical insertion tip 56, and two or four substantially-triangular or fin-like protruding structures 52 that have sharp surfaces or edges for engaging the articulating surfaces 13, 15, although other suitable assemblies are contemplated herein. The facet implant device 50 has over all dimensions on the order of several millimeters, and may be made of machined allograft (i.e. bony) material, a surgically-implantable polymeric material, a surgically-implantable ceramic material, a surgically-implantable metallic material, etc. The facet implant device 50 may include one or more holes or pores 58 along its major axis and/or perpendicular to its major axis through the body 54 thereof for the impaction of another material that promotes the fusion of the superior and inferior facets 12, 14 (FIGS. 1 and 2) of the facet joint 16. In addition, the body 54 of the facet implant device 50 may have a threaded portion 59 or other attachment means for receiving one or more tools by which it is tamped into the facet joint 16 and/or rotated. Optionally, one of the tools may act as a plunger through which fusion-promoting material is introduced into the facet implant device 50.

Referring to FIG. 8, in a still further exemplary embodiment of the present invention, the facet implant device 60 includes one or more protruding structures 62 or friction surfaces disposed concentrically there about (such as threads or the like) that engage the articulating surfaces 13, 15 (FIGS. 1 and 2) of the facet joint 16 (FIGS. 1 and 2) when the facet implant device 60 is rotated in the facet joint 16, the protruding structures 62 first contacting the articulating surfaces 13, 15, then grabbing them, then translating/distracting them with respect to one another, and then holding them securely in this translated/distracted configuration. In the embodiment illustrated, the facet implant device 60 includes a substantially-conical body 64 and a plurality of protruding structures 62 that have sharp surfaces or edges for engaging the articulating surfaces 13, 15, although other suitable assemblies are contemplated herein. The facet implant device 60 has over all dimensions on the order of several millimeters, and may be made of machined allograft (i.e. bony) material, a surgically-implantable polymeric material, a surgically-implantable ceramic material, a surgically-implantable metallic material, etc. The facet implant device 60 may include one or more holes or pores 66 along its major axis and/or perpendicular to its major axis through the body 64 thereof for the impaction of another material that promotes the fusion of the superior and inferior facets 12, 14 (FIGS. 1 and 2) of the facet joint 16. In addition, the body 64 of the facet implant device 60 may have a threaded portion or other attachment means for receiving one or more tools by which it is tamped into the facet joint 16 and/or rotated.

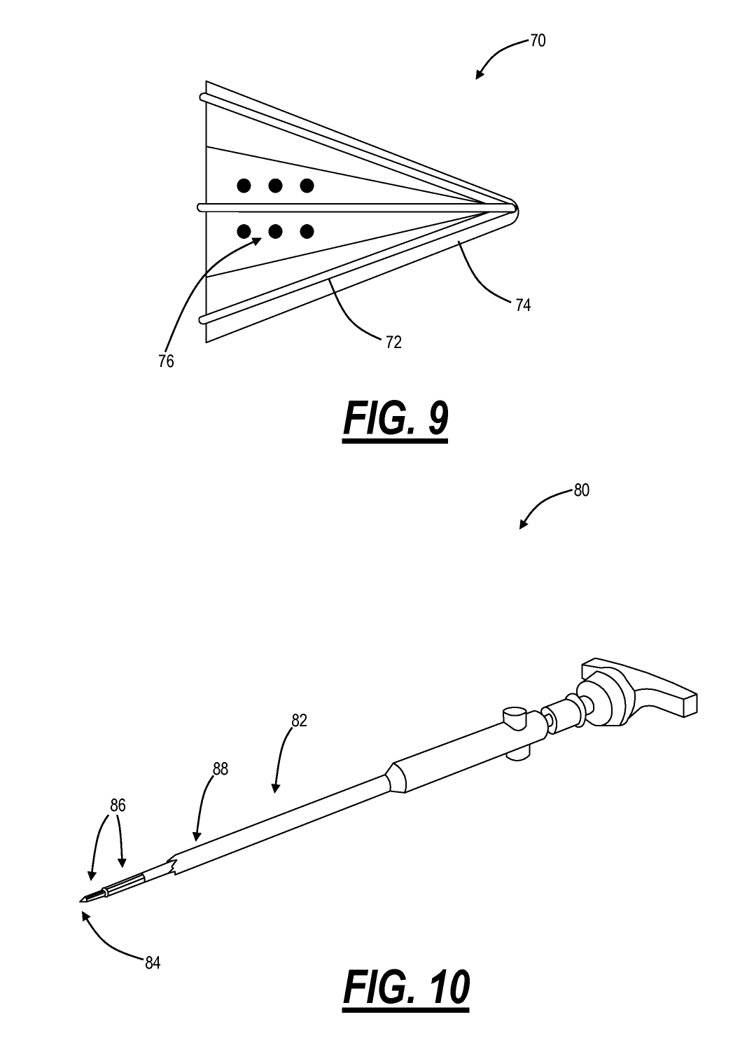

Referring to FIG. 9, in a still further exemplary embodiment of the present invention, the facet implant device 70 includes one or more protruding structures 72 or friction surfaces disposed axially there about (such as ridges or the like) that engage the articulating surfaces 13, 15 (FIGS. 1 and 2) of the facet joint 16 (FIGS. 1 and 2) when the facet implant device 70 is rotated in the facet joint 16, the protruding structures 72 first contacting the articulating surfaces 13, 15, then grabbing them, then translating/distracting them with respect to one another, and then holding them securely in this translated/distracted configuration. In the embodiment illustrated, the facet implant device 70 includes a substantially-conical body 74 and a plurality of protruding structures 72 that have sharp surfaces or edges for engaging the articulating surfaces 13, 15, although other suitable assemblies are contemplated herein. The facet implant device 70 has over all dimensions on the order of several millimeters, and may be made of machined allograft (i.e. bony) material, a surgically-implantable polymeric material, a surgically-implantable ceramic material, a surgically-implantable metallic material, etc. The facet implant device 70 may include one or more holes or pores 76 along its major axis and/or perpendicular to its major axis through the body 74 thereof for the impaction of another material that promotes the fusion of the superior and inferior facets 12, 14 (FIGS. 1 and 2) of the facet joint 16. In addition, the body 74 of the facet implant device 70 may have a threaded portion or other attachment means for receiving one or more tools by which it is tamped into the facet joint 16 and/or rotated.

It should be noted that, in all of the above embodiments, the articulating surfaces 13, 15 (FIGS. 1 and 2) of the facet joint 16 (FIGS. 1 and 2) may be cut, chiseled, gouged, or otherwise formed to substantially conform to the various surfaces of the various facet implant devices. The facet implant devices may also be advanced into the facet joint 16 upon rotation, or may be inserted, rotated to perform translation/distraction, and then further inserted to lock the facet joint 16. Any combination of elements/steps is possible.

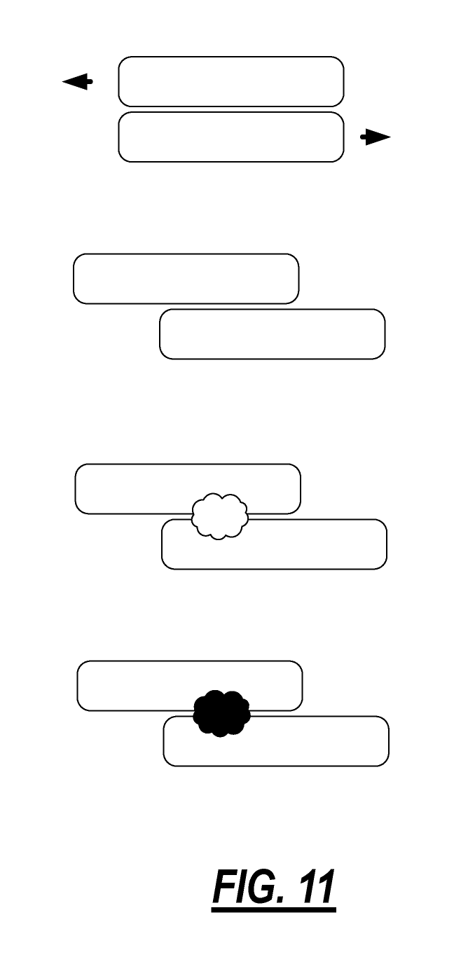

FIG. 10 is a perspective diagram illustrating one exemplary embodiment of the facet implant device tool assembly 80 of the present invention. An elongated device 82 with a sharp point 84 and a plurality of concentrically-arranged friction structures 86, for example, is inserted into the facet joint 16 (FIGS. 1 and 2) and rotated, thus providing translation/distraction. A retention sleeve 88 is then slid down the elongated device and into or adjacent to and engaging the facet joint 16 to maintain the facet joint 16 in translation/distraction while the elongated device 82 is removed. Subsequently or alternatively, a hole is drilled between and across the articulating surfaces 13, 15 (FIGS. 1 and 2) of the facet joint 16 through the retention sleeve 88 and a plug or other novel surgical implant device is tamped into the hole to maintain the facet joint 16 in translation/distraction. This later function may be accomplished using the retention sleeve 88 itself, in the case that it is simply a toothed retention washer or the like. Alternatively, the novel surgical implant device may be inserted into the facet joint 16, rotated to translate/distract the facet joint, and then remain in place itself (optionally after additional seating) to hold the facet joint 16 in the desired configuration. This surgical implant device may be a detachable end portion of the elongated device 82, for example. Alternatively, after translating/distracting, a stellate/snowflake-shaped (or other patterned) tamp may be impacted into and across the facet joint 16 to create an outline for a serrated surgical implant device to subsequently be impacted into this outline. This provides an interference fit and prevents unwinding of the facet joint 16. This facet translation/fusion method is illustrated in FIG. 11.

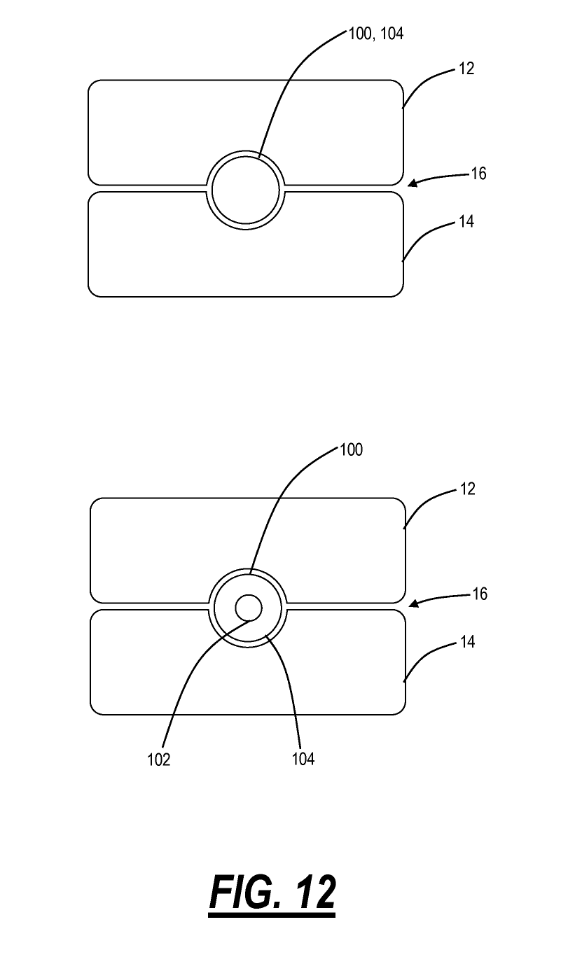

Referring to FIG. 12, in a still further exemplary embodiment of the present invention, the facet implant device 100 is disposed between the superior facet 12 and the inferior facet 14 of the facet joint 16 of a spine, optionally after prior translation/distraction of the facet joint 16. The facet implant device 100 includes a polymeric material 104 or the like (i.e. not necessarily a polymer) that is injected into the drilled or formed hole or otherwise prepared or unprepared space as a liquid or semi-solid and then rapidly hardens into a solid, thereby securely holding the facet joint 16 in its present configuration. Optionally, the polymeric material 104 or the like is disposed about and/or through a solid implant 102 or other solid device that is inserted into the drilled or formed hole or otherwise prepared or unprepared space previously or subsequently. Again, the facet implant device 100 has over all dimensions on the order of several millimeters, and the solid portion (if used) may be made of machined allograft (i.e. bony) material, a surgically-implantable polymeric material, a surgically-implantable ceramic material, a surgically-implantable metallic material, etc. The solid portion of the facet implant device 100 (if used) may include one or more holes or pores along its major axis and/or perpendicular to its major axis through the body thereof for the impaction of another material that promotes the fusion of the superior and inferior facets 12, 14 of the facet joint 16. In addition, the body of the solid portion of the facet implant device 100 (if used) may have a threaded portion or other attachment means for receiving one or more tools by which it is tamped into the facet joint 16 and/or rotated.

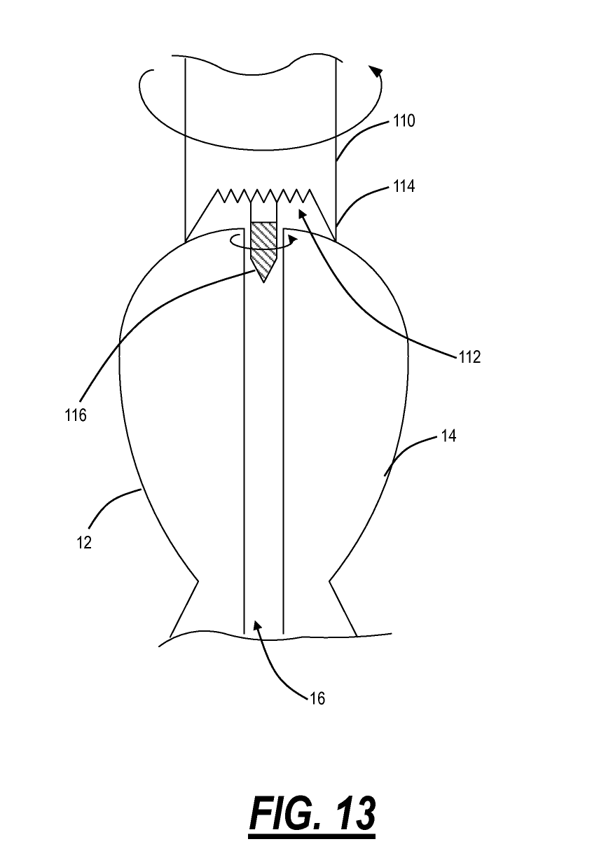

Referring to FIG. 13, in a still further exemplary embodiment of the present invention, a translation sheath 110 is provided that aides in the translation of the superior and inferior facets 12 and 14 of a facet joint 16, as is described in other related embodiments of the present invention. For example, this translation sheath 110 may be used in conjunction with the facet implant device tool assembly 80 of FIG. 10. The translation sheath 110 includes a plurality of raised teeth, serrations, or other friction structures 112 disposed around its circumference. Two or more of these raised teeth, serrations, or other friction structures 112 include spike structures 114, optionally disposed about 180 degrees from each other about the circumference of the translation sheath 110. These spike structures 114 are configured to securely engage the superior and inferior facets 12 and 14, such that the translation sheath 110 may be manually rotated, thereby translating the superior and inferior facets 12 and 14. A rotation device 116 is selectively extended from within the translation sheath 110, and is also operable for achieving purchase with and rotating the facet joint 16, as is also described in other related embodiments of the present invention. This extension may occur before and/or after the translation sheath 110 is advanced onto the facet joint 16. Optionally, the rotation device 116 is used to translate the facet joint 16, and then the translation sheath 110 is tapped into place and used to further translate the facet joint 16. Thus, these rotations may be complementary. It will be understood by those of ordinary skill in the art that the translation sheath 110, or a similar facet joint engagement and translation device, may also be used alone, without a device such as the rotation device 116 or other device, to engage and translate or distract the facet joint 16. Likewise, it will be understood by those of ordinary skill in the art that the rotation device 116, or a similar facet joint engagement and translation device, or importantly a facet joint implant itself, may also be used alone, without a device such as the translation sheath 110 or other device, to engage and translate or distract and hold the facet joint 16. In this sense, parts of all of the embodiments of the present invention may be used selectively with parts of other embodiments.

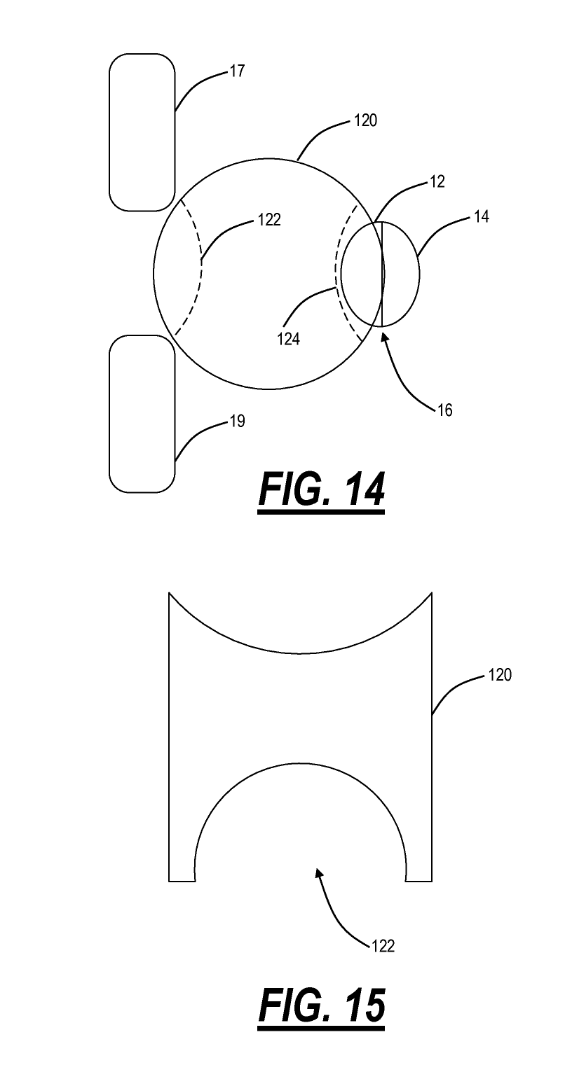

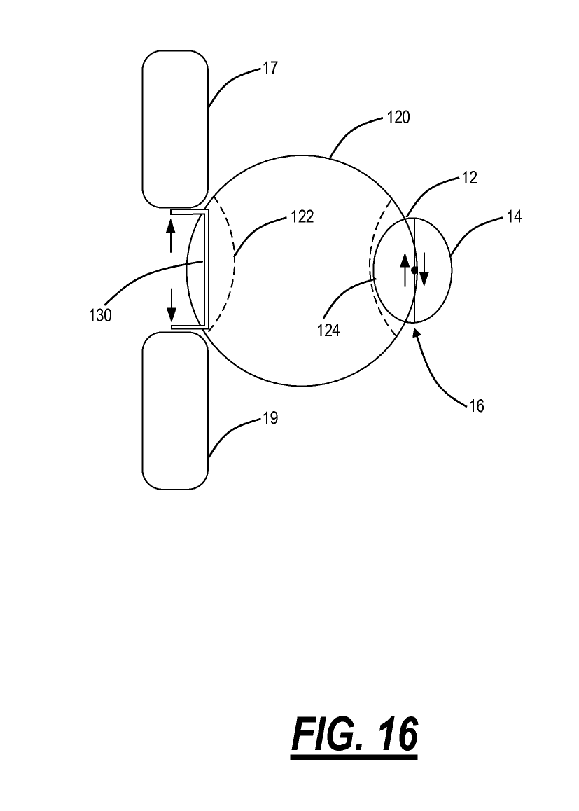

Referring to FIGS. 14-16, in a still further exemplary embodiment of the present invention, a facet portal system 120 is provided that provides surgical access to adjacent spinous processes 17 and 19 such that they may be distracted, as well as the associated facet joint 16 such that it may be translated and or fused as taught through this disclosure. This essentially allows the associated disc to be unloaded and a partial or total posterior discectomy to be performed in conjunction with a facet fusion. The facet portal system 120 is selectively disposed in the triangular region between the adjacent spinous processes 17 and 19 and the associated facet joint 16. An arch-shaped cut-away 122 or the like disposed on one side of the lower portion of the facet portal system 120 provides surgical access to the adjacent spinous processes 17 and 19, while, optionally, an arch-shaped cut-away 124 or the like disposed on the other side of the lower portion of the facet portal system 120 provides surgical access to the associated facet joint 16 (such access may, however, be available without the use of the arch-shaped cut-away 124 or the like, either through the bottom or outside of the facet portal system 12, for example). Once the facet portal system 120 is in place, a distractor 130 is placed through the facet portal system 120 and into the interspinous process space, and the adjacent spinous processes 17 and 19 are distracted. This unloads the disc and translates the facet joint 16. At this point, the facet joint 16 is drilled or otherwise prepared as described throughout this disclosure and the distractor 130 is removed. Next, a partial posterior discectomy is performed and the distractor 130 is replaced and distraction is restored, again unloading the disc and translating the facet joint 16. At this point, a facet implant is placed to fuse the facet joint 16 as described throughout this disclosure, also maintaining the spinous process distraction. Finally, the distractor 130 and facet portal system 120 are removed. It will be understood by those of ordinary skill in the art that the ordering of these steps may be varied. For example, a partial or total discectomy may be performed at any point, and may be omitted.

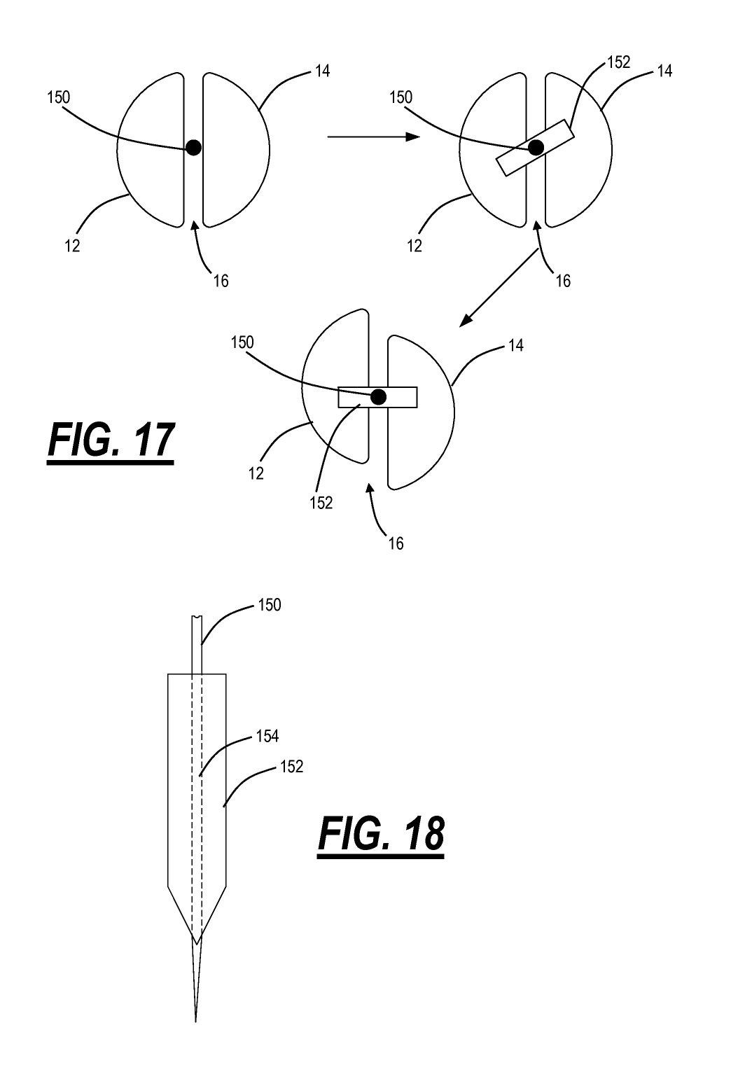

Referring to FIGS. 17 and 18, in a still further exemplary embodiment of the present invention, a rigid or semi-rigid needle or post 150 is selectively disposed between the superior facet 12 and the inferior facet 14 of the facet joint 16 of a spine and acts to localize the facet joint 16. Am appropriately rigid wire 150 could also be used for this purpose equally. Subsequently, a rigid or semi-rigid cannulated keel structure 152 having a substantially-planar or other suitable configuration and a sharpened lower edge is guided over the needle or post 150 into engagement with the superior facet 12 and the inferior facet 14, and impacted into or otherwise engaged with the two halves of the facet joint 16, optionally at an offset angle relative to the two halves of the facet joint 16. Accordingly, it will be readily apparent to those of ordinary skill in the art that the keel structure 152 may have a variety of shapes, sizes, and configurations suitable for securely engaging the facet joint 16. Once in place, the keel structure 152 is rotated, thereby translating the facet joint 16, which is optionally also held in this translated state by any of the means provided in the present disclosure. It will be understood by those of ordinary skill in the art that the keel structure 152 or other similar device does not have to be cannulated, and may be used without the needle, post, or wire 150 equally. Further, the keel structure 150 may only engage a facet 12 or 14 on one side of the facet joint 16, thereby moving that facet 12 or 14 with respect to the other facet 12 or 14, which is left essentially unmoved. In this sense, the keel structure 152 could be a one-sided structure. Still further, the keel structure 152 may not be impacted into the facet(s) 12 and/or 14, but may simply engage a roughened or otherwise prepared surface of one or both. This equivalence, between cut-away, roughened, and otherwise prepared surfaces of the bony structures involved applies equally throughout all embodiments of the present invention—all of these terms being defined as such surfaces being prepared for accepting engagement with the various devices and implants.

Although the present invention is illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A surgical device for the translation/distraction and subsequent stabilization/fusion of a facet joint of a spine, comprising:
a guide that is selectively disposed partially between articulating surfaces of the facet joint; and
a keel structure that is selectively disposed about the guide and engaged with the articulating surface(s) of one or more of the facet joint(s), wherein, when the keel structure is selectively rotated about the guide, the articulating surfaces of the facet joint are moved with respect to one another.

2. The surgical device of claim 1, wherein the keel structure has a substantially planar configuration.

3. The surgical device of claim 1, wherein the keel structure has a sharpened lower portion.

4. A surgical system for the translation/distraction and subsequent stabilization/fusion of a facet joint of a spine, comprising:
a portal tube defining a first cut-away portion, wherein, when placed, the first cut-away portion provides surgical access to adjacent spinous processes of the spine.

5. The surgical system of claim 4, wherein the portal tube further defines a second cut-away portion disposed substantially opposite the first cut-away portion, wherein, when placed, the second cut-away portion provides surgical access to an associated facet joint of the spine.

6. The surgical system of claim 4, further comprising a distraction device that is selectively disposed within the tube, through the first cut-away portion, and between the adjacent spinous processes.

7. The surgical system of claim 5, further comprising one of a facet joint implant and device that is selectively disposed within the tube, through the second cut-away portion, and within/by one of a receiving hole and a receiving surface formed in/on the facet joint.

8. A surgical method for the translation/distraction and subsequent stabilization/fusion of a facet joint of a spine, comprising:
through a portal tube, disposing a distraction device between adjacent spinous processes of the spine and distracting the adjacent spinous processes;
one or more of forming a hole across the facet joint of the spine and preparing surfaces of the facet joint of the spine; and
inserting one or more of a facet implant and a facet device one or more of in the hole formed across the facet joint or in engagement with the prepared surfaces of the facet joint.

9. The surgical method of claim 8, further comprising performing a discectomy one of before and after distracting the adjacent spinous processes.

10. The surgical method of claim 9, further comprising replacing the distraction device and again distracting the adjacent spinous processes.

11. A surgical device for the translation/distraction and subsequent stabilization/fusion of a facet joint of a spine, comprising:
a rotation device that is selectively disposed at least partially between and engages articulating surfaces of the facet joint, wherein the rotation device is operable for rotating and translating the articulating surfaces of the facet joint; and
a translation sheath disposed about the rotation device that is selectively mated with a superior facet and an inferior facet of the facet joint, wherein the translation sheath is operable for rotating and further translating the articulating surfaces of the facet joint.

12. The surgical device of claim 11, wherein the translation sheath comprises a plurality of raised structures disposed about a circumference thereof.

13. The surgical device of claim 11, wherein the translation sheath comprises a plurality of spike structures disposed about a circumference thereof.

14. A surgical implant device for the stabilization/fusion of a facet joint of a spine, comprising:
a liquid or semi-solid material that is disposed one or more of in a cut-away portion of each articulating surface of the facet joint and adjacent to a prepared portion of each articulating surface of the facet joint and subsequently allowed to harden to a solid to prevent movement thereof.

15. The surgical implant device of claim 14, further comprising a solid material that is disposed within or about the liquid or semi-solid material one or more of within the cut-away portion of each articulating surface of the facet joint and adjacent to the prepared portion of each articulating surface of the facet joint to prevent movement thereof.

16. The surgical implant device of claim 14, wherein the liquid or semi-solid material is placed through a portal tube.

17. The surgical implant device of claim 15, wherein the solid material is placed through a portal tube.
